(19) United States
(12) Patent Application Publication
(10) Pub. No.: US 2025/0391532 A1
(43) Pub. Date: Dec. 25, 2025

(54) DYNAMIC ANTI-CANCER DRUG RESPONSE PREDICTIVE METHOD AND MACHINE LEARNING SYSTEM WITH MOLECULAR BIOLOGY AS THE CORE AND INTEGRATED MULTIPLE EPIGENETIC FACTORS

(71) Applicant: Xinyue Zhou, Haikou (CN)

(72) Inventor: Xinyue Zhou, Haikou (CN)

(21) Appl. No.: 18/747,812

(22) Filed: Jun. 19, 2024

(51) Int. Cl. G16H 20/10; G16B 5/00; G16B 20/10; G16B 20/20; G16B 20/50; G16B 40/20; G16B 40/30; G16H 10/60

(52) U.S. Cl. CPC G16H 20/10; G16B 5/00; G16B 20/10; G16B 20/20; G16B 20/50; G16B 40/20; G16B 40/30; G16H 10/60

(57) ABSTRACT

The present invention discloses a dynamic anti-cancer drug response predictive method and machine learning system with molecular biology as the core and integrated multiple epigenetic factors. More specifically, the present invention integrates multiple epigenetic data, predicts based on the central dogma, and builds a dynamic model. The system directly inputs the data from the cloud, accurately locates, and automatically pre-processes the data in the algorithm-required form. The disclosed method and machine learning system are always open and continuously integrate the latest scientific progress to achieve dynamic implementation. Combined with the targeted needs of cancer prediction, relevant algorithms that can highlight the characteristics of the data set are selected to allow the biological model to play the greatest leading role. Unsupervised and supervised algorithms are selected to be jointly constructed, and users can combine the conclusions of the two to draw answers jointly.

### TECHNICAL FIELD

The present invention relates to the field of auxiliary detection technology, and specifically to a dynamic anti-cancer drug response predictive method and machine learning system with molecular biology as the core and integrated multiple epigenetic factors.

### BACKGROUND

Cancer poses a significant threat to human health and its complex dynamics involve various molecular biological mechanisms. The current limitations in cancer diagnosis and treatment methods emphasize the need for a more sustainable, efficient, and personalized approach. To address these challenges, the proposed method leverages the continuous advancements in molecular biology technology and the emergence of epigenetics. Epigenetic modification, which includes mechanisms such as DNA methylation and histone modification, has been identified as closely linked to the occurrence and development of cancer. By studying the interplay between epigenetic modification and cancer, this method seeks to introduce innovative pathways for the selection of anticancer drugs and treatment methods.

Moreover, the method integrates multiple epigenetic factors with molecular biological insights to build a dynamic predictive model for cancer. Through the synergy of machine learning algorithms and epigenetic knowledge, this model is aimed at offering a comprehensive and individualized approach to cancer treatment. The proposed method envisions a paradigm shift in cancer treatment, enhancing the efficiency and accuracy of interventions based on personalized biological characteristics.

### SUMMARY

The purpose of the present invention is to overcome the above problems and provide a method and a dynamic anti-cancer drug response predictive machine learning system with molecular biology as the core and integrated multiple epigenetic factors. To achieve the above purpose, the present invention adopts the following technical solutions:

A method and a dynamic anti-cancer drug response predictive machine learning system with molecular biology as the core and integrated multiple epigenetic factors., including the following steps:

### S1. Constructing a Biological Model

Based on the core of molecular biology, the main structure of DNA-RNA-Protein of central dogma, six epigenetic factors are integrated, namely: Copy Number Variants (CNVs), Damaging Mutations, RNA interference (RNAi), DNA Global methylation, Protein arrays and Proteomics.

The construction of biological models is divided into two logical frameworks:

One is the construction of the core idea of molecular biology, that is, DNA is transcribed into RNA, and then translated into protein, to build a causal relationship. The six epigenetic factors are respectively related to DNA, RNA and Protein. That is, Copy Number Variants (CNVs) and damaging mutations have a direct and only DNA-related impact. Global methylation of whole genome DNA, RNA interference RNAi, has a strong connection with RNA, and is related to both DNA transcription and DNA translation.

Global DNA Methylation is divided into two parts, one is hyper methylation and the other is hypo methylation. Hypo Methylation has the function of gene regulation, thus affecting transcription and translation. Hyper methylation and siRNA and RISC in RNAi all play an important role in gene silencing. This process affects DNA transcription and obtains different RNAs. On the other hand, miRNA in RNAi directly affects the translation process and has a connectivity effect in the middle layer of DNA-RNA-Protein. Proteomics and protein arrays are a deep understanding and expansion of the last layer of protein. One takes factors such as dosage into consideration, and the other takes detection methods into consideration. Multiple tests are performed to obtain accurate information and make full preparations for protein analysis.

The second logical framework is the relationship between various epigenetics.

This can be divided into two levels. One is correlation, which is a joint action relationship. Hyper methylation and siRNA in RNAi, RISC all play an important role in gene silencing, jointly affecting DNA transcription and obtaining different RNAs. Among them, the protein arrays data set contains RPPA data, and RPPA itself is both a proteome technology and a special protein array, which has shown the relationship between the two. Structural genomics and proteomics will lead the exploration of protein expression systems. On the other hand, protein arrays and proteomics link genomics and proteomics, which can be used for cancer treatment and related drug development.

The second level is causality, which is not a strong biological causal relationship, but a narrow causality, that is, the change of a certain factor leads to the change of another factor. CNVs can compare DNA methylation. When the copy number increases, CNVs will cause deviations in the measurement of DNA methylation. Specifically, when one allele is lost, a small deviation may occur, and when both alleles are lost, a large deviation may occur. Methylation plays a role in gene silencing and regulation in gene expression, as exemplified by the fact that the aprt gene is repressed by CpG methylation in the 5′ region, but is not affected by methylation at the 3′ end or adjacent M13 sequences. Modifications in how the expression of some specific genes (particularly transcription factors) is associated with DNA methylation variation in a tissue-dependent manner. Methylation (hyper methylation and hypo methylation) has been shown to have a driving effect on proteomics. It has been shown above that CNVs have a direct effect on methylation, and methylation has a direct effect on proteomics and protein arrays. Disruptive mutations may affect the RNA interference (RNAi) pathway. Through knockout studies, the effects of RNAi on Protein Arrays and Proteomics can be discovered.

S2. Write biological models into algorithms and express the core of molecular biology using computer languages and mathematical models;

Correlation combines antagonism and synergy in biological models into joint action. It can be described in more detailed language as a biological relationship in which a factor acts together with another factor or affects another factor.

Causal is a narrow causal relationship in biological models, that is, a certain factor affects another factor. The core relationship in molecular biology is the process of DNA-transcription-RNA-translation-Protein, which can be summarized as a causal relationship.

The sum of the previous biological relationships is written and fitted into the program in an algorithmic language, and the two relationships are analyzed and written into the algorithm. List them out:

Correlation analysis: CNVs and Damaging mutations, Global DNA Methylation and RNAi, Proteomics and Protein Arrays.

Causal analysis: (CNVs and Damaging mutations) this whole layer and (Global DNA Methylation and RNAi), (Global DNA Methylation and RNAi) this whole layer and (Proteomics and Protein Arrays).

### S3. Data Processing

The data preprocessing in this paper is divided into two parts, one is general data processing and the other is specialized data preprocessing.

### 1. General Data Processing

The specific implementation of general data preprocessing is divided into two python files. Since the original data does not give all the indexes, incomplete index rows are added first and then the missing value processing is performed. And since the original data set does not have a real index column, an auxiliary column is added actively, which will be deleted after completing the index row and integrating the data set with the corresponding biological level.

The functions of the two python files are 1. Complete the discontinuous index to create a data set with the same index 2. Horizontally concatenate the DataFrames within each level to create merged datasets for each level. According to different processing requirements, the two files use different ways of loading documents. One method uses the “glob” function in the “pathlib” library, which returns a generator for later iteration. The other method uses the “glob” function in the “glob” library, which returns a list.

### 2. Specialized Data Preprocessing

The specialized data processing phase is tailored to suit the algorithms. There are two algorithms: one is the supervised learning Wide & Deep algorithm, and the other is the unsupervised learning Mini Batch Kmeans algorithm.

### Supervised Wide & Deep

It starts with a specialized preprocessing phase, which includes deleting all columns with the same name as the first column, resetting the index if it contains unique values, dropping columns with all NaN values, converting the DataFrame to a sparse format, and dropping columns with the same name as the first row.

The data is reshaped if it does not contain dimensions of size 0. If the DataFrame has no rows or columns, reshaping is skipped. Otherwise, it flattens the DataFrame, takes a certain number of elements, reshapes them into a specific array, and then converts it back to a Dask DataFrame.

The next steps include loading two datasets, preprocessing them and use static hold-out method to split dataset and process order. This process is also applied when loading a single dataset.

### Unsupervised Mini Batch Kmeans

Loads the CSV file into a Dask DataFrame, sets a specific column as the index, drops columns with all NaN values, normalize the data, converts the DataFrame to a sparse format, and drop columns that are the same as the first row

### S4. Model Building

The above model structure is integrated into the algorithm implementation part. Due to the feature of medical data and biological data, both supervised learning and unsupervised learning can be used. Therefore, two models are used in this system. Supervised learning uses the Wide & Deep algorithm, and unsupervised learning uses the Mini Batch Kmeans algorithm.

### Supervised Wide & Deep

The biological model is first integrated into the algorithm by performing correlation analysis to highlight different biological features that collaborate in the same biological activity. After the correlation analysis, the causal relationship between the causal biological factors is processed. The causal effect is estimated using a stochastic gradient descent (SGD) regressor, while a parallel process is used to improve efficiency.

A hybrid approach Wide & Deep algorithm is used to process different types of features and provide a balance between memory and generalization. All attributes are automatically initialized in the main block. The wide component is a double linear, takes user (patient's cell line) and item (anti-cancer drug) as input, transforms the input features, and inputs the data into a two-layer linear layer. The deep component is two feed forward neural networks that learn high-level feature interactions. One is for the correlation relationship at the biological relationship level, and the other is for the causal relationship. It takes the output of the data results processed by each layer of biological relationship and the user and item data sets as input, applies the ReLU activation function, and passes them to more layers. The entire deep component has a total of four hidden layers. The deep correlation component organizes three datasets and user and item datasets, with a total of three hidden layers. But because no connection can be found for gene mutations, which is natural, the deep causal component has only one hidden layer. During the forward pass, the input passes through a double linear wide component and two deep components, and the outputs of the three components are added to produce the final output.

For each epoch, the cell lines, drugs, and labels are converted to the specific data types required by the help function, and the training data loader is iterated to generate all the data. The model then outputs predictions, which are reshaped if necessary to match the shape of the labels. The loss between the model output and the actual labels is calculated using the MSE loss criterion. The gradients are then back propagated through the model and the model's parameters are updated using the Adam optimizer. After the model is trained, it is validated using the hold-out processed test dataset. The test data is converted to tensor form and the cell line test data is unpacked if necessary to ensure it has the correct dimensions. The test data loader is then iterated and yielded again. The model makes predictions based on the cell line and drug, and the loss between these predictions and the actual labels is calculated.

### Unsupervised Mini Batch Kmeans

Use causal and correlation analysis to write the previous biological model and convert it into a data format that the algorithm can accept then preprocess missing values, add garbage collection to free the memory of unused or unreferenced objects in the Python memory management system.

Modify the unsupervised model by extracting the results of the previous causality and correlation analysis, use this to filter rows, apply the Mini Batch KMeans algorithm, calculate the silhouette score to measure the similarity of the object to its own cluster compared to other clusters, and use PCA (Principal Component Analysis) for dimensionality reduction and visualization, which can handle prediction work. The optimal number of clusters is dynamically determined by plotting the relationship between inertia (the sum of the squared distances from the sample to the nearest cluster center) and the number of clusters.

The optimal number of clusters is determined by elbow and automatically integrated, making it fully automatic and dynamic.

### BRIEF DESCRIPTION OF THE DRAWINGS

FIG. 1 is a biological model of the relationships between epigenetic factors.

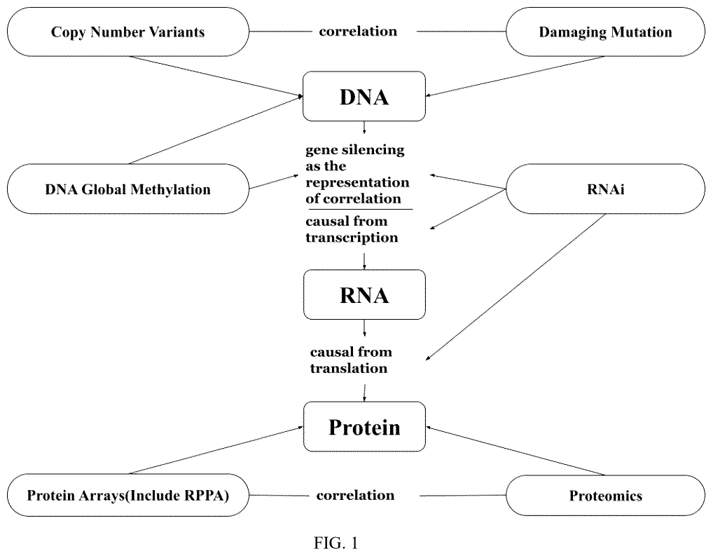

FIG. 2 is the general data processing flow.

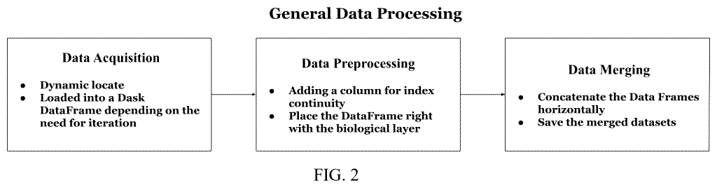

FIG. 3 is the specialized data processing flow embedded in the algorithm.

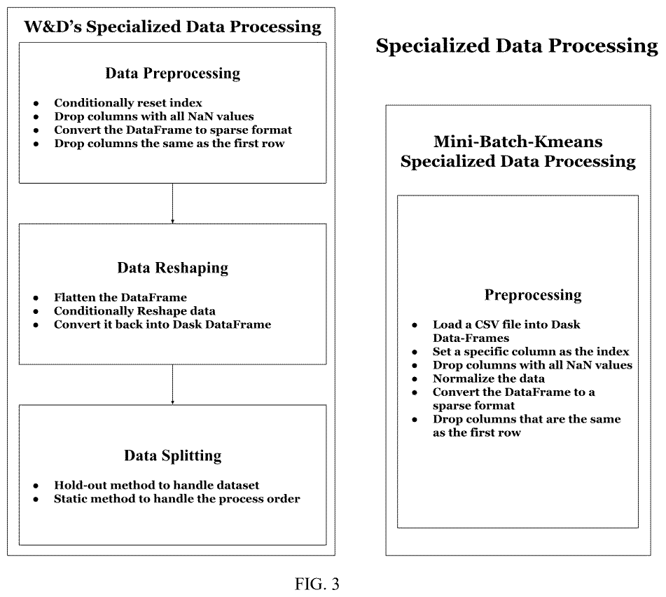

FIG. 4 is a schematic diagram of the specific hierarchical relationship of the Wide & Deep algorithm model.

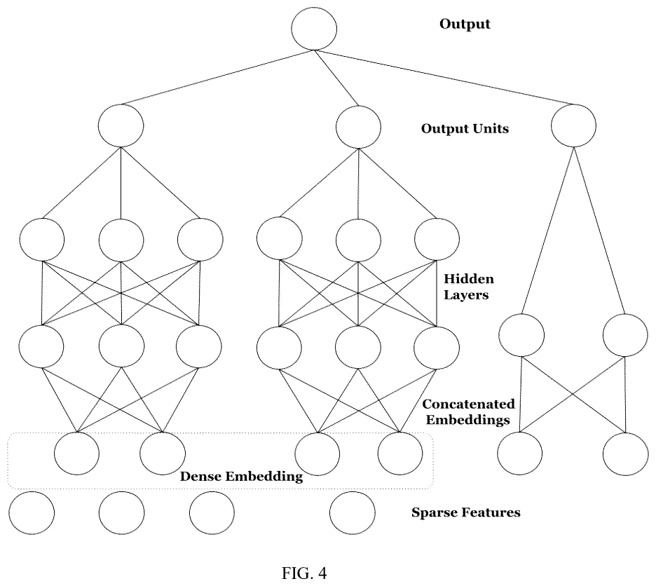

FIG. 5 is a flowchart of the supervised learning model.

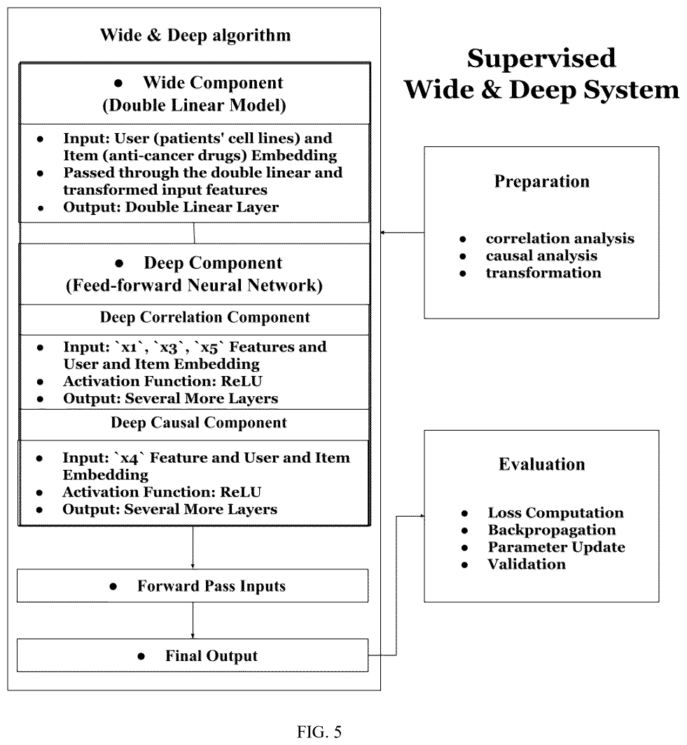

FIG. 6 is a flowchart of the unsupervised learning model.

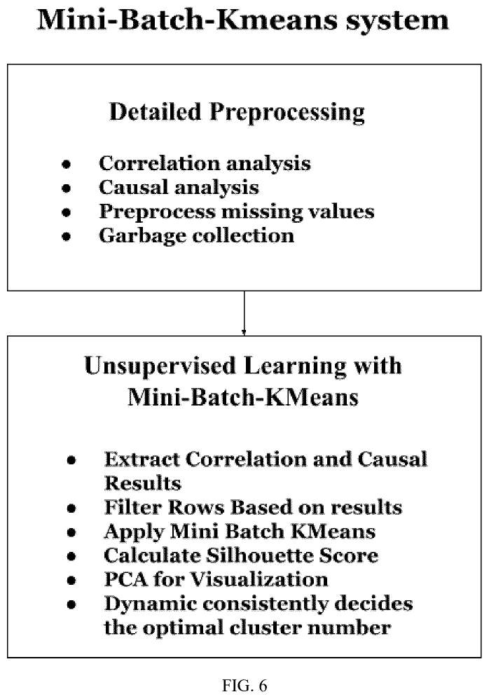

### DETAILED DESCRIPTION OF THE DISCLOSED EMBODIMENTS

In order to make the purpose, technical solutions and advantages of the embodiments of the present invention clearer, the technical solutions in the embodiments of the present invention will be clearly and completely described below in conjunction with the drawings in the embodiments of the present invention. Obviously, the described embodiments are part of the embodiments of the present invention, not all of the embodiments. Generally, the components of the embodiments of the present invention described and shown in the drawings here can be arranged and designed in various different configurations.

### Embodiment 1

This embodiment discloses a dynamic anti-cancer drug response predictive method with molecular biology as the core and integrated multiple epigenetic factors, characterized by comprising the following steps:

S1. Constructing a biological model
S1-1. Constructing a causal relationship at the core concept level of molecular biology;

Epigenetic factors used in this example include: Copy Number Variants (CNVs), Damaging Mutations, RNA interference (RNAi), DNA Global methylation, Protein arrays and Proteomics.
the construction of relationships at the molecular biology core concept level includes:
S1-1-1. Construct the relationship between Copy Number Variants (CNVs) and Damaging Mutations at the DNA level;
S1-1-2. Construct the relationship between RNA interference (RNAi) and DNA Global methylation at the RNA level;
S1-1-3. Construct the relationship between Protein arrays and Proteomic at Protein level;
S1-2. Constructing correlations between epigenetic factors
the correlation relationship is to combine the antagonism and synergy in the biological model into a common effect, including:
S1-2-1. Construct the correlation relationship between Copy Number Variants (CNVs) and Damaging Mutations;
S1-2-2. Construct the correlation relationship between RNA interference (RNAi) and DNA Global methylation;
S1-2-3. Construct the correlation relationship between Protein arrays and Proteomics;

The correlation relationship is obtained by calculating the Pearson coefficient.
S1-3. Constructing causal relationship between epigenetic factors

A causal relationship is when a change in one of the epigenetic factors affects another factor;
S1-3-1. Construct the causal relationship between [Copy Number Variants (CNVs) and Damaging Mutations] and [RNA interference (RNAi) and DNA Global methylation];
S1-3-2. Construct the causal relationship between [RNA interference (RNAi) and DNA Global methylation] and [Protein arrays and Proteomics];

The causal relationship is constructed using SGD Regression in supervised learning, and quadratic sample interpolation is used in unsupervised learning to construct the causal relationship.

S2. Write biological models into algorithms and express the core of molecular biology using computer languages and mathematical models;

S3. Data processing
S3-1. General data processing;
   Receive patient data in real time, receive total cloud data sets in real time, and automatically locate file addresses.
   3-1-1. Use the ‘pathlib’ to dynamically locate the file loading address;
   3-1-2. Use the ‘glob’ function in the ‘pathlib’ library to return a generator for later iteration;
   3-1-3. Use the ‘glob’ function in the ‘glob’ library, which returns a list;
   3-1-4. Iterate over the csv files in the generator, and create datasets with the same index by adding a column of ‘num’ to complete the discontinuous indexes;
   3-1-5. Process the csv files in the list, and the processed datasets are integrated at the biological level for data merging;
S3-2. Specialized data preprocessing for Wide & Deep algorithms;
   3-2-1. Delete all columns with the same name as the first column and reset the index if it contains unique values;
   3-2-2. Drop columns with all NaN values
   3-2-3. Convert the DataFrame to sparse format Drop columns the same as the first row;
   3-2-4. Flatten the DataFrame to get a certain number of elements;
   3-2-5. Conditionally reshape data convert it back into Dask DataFrame;
   3-2-6. Hold-out method to handle dataset Static method to handle the process order
S3-3. Specialized data preprocessing for Mini Batch Kmeans algorithms;
   3-3-1. Load a CSV file into Dask Data-Frames
   3-3-2. Set a specific column as the index
   3-3-3. Drop columns with all NaN values
   3-3-4. Normalize the data
   3-3-5. Convert the DataFrame to a sparse forma
   3-3-6. Drop columns that are the same as the first row

S4. Model building
S4-1. Building a supervised model;
   4-1-1. Use causal and correlation analysis to write the previous biological model and convert it into a data format acceptable to the algorithm, use stochastic gradient descent (SGD) regressor to estimate causal effects, and use parallel processes to improve efficiency;
   4-1-2. Wide Component with feature interactions take the user (patient's cell line) and project (anti-cancer drug) as input, change the input features, and pass the data to the generalized linear model with two-layer feature interactions;
   4-1-3. Deep correlation component organizes three data sets and user and item data sets;
   4-1-3. Deep correlation uses Relu activation;
   4-1-4. Deep correlation is passed to more layers;
   4-1-5. Deep causal component has only one hidden layer, and the rest is the same as 4-1-3.4-1-4;
   4-1-6. Forward Pass Inputs;
   4-1-7. Final Output;
   4-1-8. Use the MSE loss criterion to calculate the loss between the model output and the actual label;
   4-1-9. Back propagation;
   4-1-10. Use Adam optimizer to update the parameters of the model;
   4-1-11. Validation;
S4-2. Building an unsupervised model.
   4-2-1. Use causal and correlation analysis to write the previous biological model and convert it into a data format that the algorithm can accept;
   4-2-2. Preprocess missing values
   4-2-3. Garbage collection
   4-2-4. Extract Correlation and Causal Results
   4-2-5. Filter Rows Based on results
   4-2-6. Apply Mini Batch KMeans
   4-2-7. Calculate Silhouette Score
   4-2-8. PCA for Visualization
   4-2-9. Dynamic consistently decides the optimal cluster number

The specific embodiments of the present invention are described in detail above, but they are only examples, and the present invention is not equivalent to the specific embodiments described above. For those skilled in the art, any equivalent modifications and substitutions made to the present invention are also within the scope of the present invention. Therefore, the equalization changes and modifications made without departing from the spirit and scope of the present invention should be included in the scope of the present invention.

1. A dynamic anti-cancer drug response predictive method and machine learning system with molecular biology as the core and integrated multiple epigenetic factors, characterized by comprising the following steps:
S1. Constructing a biological model
S1-1. Constructing a causal relationship at the core concept level of molecular biology;
S1-2. Constructing a correlation relationship between epigenetic factors;
S1-3. Constructing a causal relationship between epigenetic factors;
S2. Write biological models into algorithms and express the core of molecular biology using computer languages and mathematical models;
S3. Data processing
S3-1. General data processing;
S3-2. Specialized data preprocessing for Wide & Deep algorithms;
S3-3. Specialized data preprocessing for Mini Batch Kmeans algorithms;
S4. Model building
S4-1. Building a supervised model;
S4-2. Building an unsupervised model.

2. The dynamic anti-cancer drug response predictive method and machine learning system with molecular biology as the core and integrated multiple epigenetic factors of claim 1, the dynamic anti-cancer drug response predictive method with molecular biology as the core and integrated multiple epigenetic factors characterized in that, the S3-1 data collection step receives patient data in real time, receives total cloud data in real time, and automatically locates the file address.

3. The dynamic anti-cancer drug response predictive method and machine learning system with molecular biology as the core and integrated multiple epigenetic factors of claim 1, the dynamic anti-cancer drug response predictive method with molecular biology as the core and integrated multiple epigenetic factors characterized in that the S4 step automatically selects the number of clusters for unsupervised learning and dynamically accepts changing biological discoveries due to the free-to-change nature of the dataset.

4. The dynamic anti-cancer drug response predictive method and machine learning system with molecular biology as the core and integrated multiple epigenetic factors of claim 1, the dynamic anti-cancer drug response predictive method with molecular biology as the core and integrated multiple epigenetic factors characterized in that Epigenetic factors include: Copy Number Variants (CNVs), Damaging Mutations, RNA interference (RNAi), DNA Global methylation, Protein arrays and Proteomics.

5. The dynamic anti-cancer drug response predictive method and machine learning system with molecular biology as the core and integrated multiple epigenetic factors of claim 4, the dynamic anti-cancer drug response predictive method with molecular biology as the core and integrated multiple epigenetic factors, characterized in that, the construction of relationships at the molecular biology core concept level described in step S1-1 includes:
S1-1-1. Construct the relationship between Copy Number Variants (CNVs) and Damaging Mutations at the DNA level;
S1-1-2. Construct the relationship between RNA interference (RNAi) and DNA Global methylation at the RNA level;
S1-1-3. Construct the relationship between Protein arrays and Proteomic at Protein level;

6. The dynamic anti-cancer drug response predictive method and machine learning system with molecular biology as the core and integrated multiple epigenetic factors of claim 4, the dynamic anti-cancer drug response predictive method with molecular biology as the core and integrated multiple epigenetic factors, characterized in that the correlation relationship in step S1-2 is to combine the antagonism and synergy in the biological model into a common effect, including:
S1-2-1. Construct the correlation relationship between Copy Number Variants (CNVs) and Damaging Mutations;
S1-2-2. Construct the correlation relationship between RNA interference (RNAi) and DNA Global methylation;
S1-2-3. Construct the correlation relationship between Protein arrays and Proteomics;
the correlation relationship is obtained by calculating the Pearson coefficient.

7. The dynamic anti-cancer drug response predictive method and machine learning system with molecular biology as the core and integrated multiple epigenetic factors of claim 4, the dynamic anti-cancer drug response predictive method with molecular biology as the core and integrated multiple epigenetic factors, characterized in that the causal relationship in step S1-3 is caused by the change of one of the epigenetic factors affecting another factor;
S1-3-1. Construct the causal relationship between [Copy Number Variants (CNVs) and Damaging Mutations] and [RNA interference (RNAi) and DNA Global methylation];
S1-3-2. Construct the causal relationship between [RNA interference (RNAi) and DNA Global methylation] and [Protein arrays and Proteomics];
the causal relationship is constructed using SGD Regression in supervised learning, and quadratic sample interpolation is used in unsupervised learning to construct the causal relationship.

8. The dynamic anti-cancer drug response predictive method and machine learning system with molecular biology as the core and integrated multiple epigenetic factors of claim 1, the dynamic anti-cancer drug response predictive method with molecular biology as the core and integrated multiple epigenetic factors, characterized in that the S3 data processing step is specifically as follows:
3-1-1. Use the ‘pathlib’ to dynamically locate the file loading address;
3-1-2. Use the ‘glob’ function in the ‘pathlib’ library to return a generator for later iteration;
3-1-3. Use the ‘glob’ function in the ‘glob’ library, which returns a list;
3-1-4. Iterate over the csv files in the generator, and create datasets with the same index by adding a column of ‘num’ to complete the discontinuous indexes;
3-1-5. Process the csv files in the list, and the processed datasets are integrated at the biological level for data merging;
3-2-1. Delete all columns with the same name as the first column and reset the index if it contains unique values;
3-2-2. Drop columns with all NaN values;
3-2-3. Convert the DataFrame to sparse format Drop columns the same as the first row;
3-2-4. Flatten the DataFrame to get a certain number of elements;
3-2-5. Conditionally reshape data and convert it back into Dask DataFrame;
3-2-6. Static hold-out method to handle dataset and process order;
3-3-1. Load a CSV file into Dask Data-Frames;
3-3-2. Set a specific column as the index;
3-3-3. Drop columns with all NaN values;
3-3-4. Normalize the data;
3-3-5. Convert the DataFrame to a sparse format;
3-3-6. Drop columns that are the same as the first row.

9. The dynamic anti-cancer drug response predictive method and machine learning system with molecular biology as the core and integrated multiple epigenetic factors of claim 1, a dynamic anti-cancer drug response predictive method with molecular biology as the core and integrated multiple epigenetic factors, characterized in that the S4 model building step is specifically as follows:
4-1-1. Use causal and correlation analysis to write the previous biological model and convert it into a data format acceptable to the algorithm, use stochastic gradient descent (SGD) regressor to estimate causal effects, and use parallel processes to improve efficiency;
4-1-2. Wide Component with feature interactions take the user (patient' s cell line) and project (anti-cancer drug) as input, change the input features, and pass the data to the double linear model;
4-1-3. Deep correlation component organizes three data sets and user and item data sets;
4-1-3. Deep correlation uses Relu activation;
4-1-4. Deep correlation is passed to more layers;
4-1-5. Deep causal component has only one hidden layer, and the rest is the same as 4-1-3. 4-1-4;
4-1-6. Forward Pass Inputs;
4-1-7. Final Output;
4-1-8. Use the MSE loss criterion to calculate the loss between the model output and the actual label;
4-1-9. Back propagation;
4-1-10. Use Adam optimizer to update the parameters of the model;
4-1-11. Validation;
4-2-1. Use causal and correlation analysis to write the previous biological model and convert it into a data format that the algorithm can accept;
4-2-2. Preprocess missing values;
4-2-3. Garbage collection;
4-2-4. Extract Correlation and Causal Results;
4-2-5. Filter Rows Based on results;
4-2-6. Apply Mini Batch KMeans;
4-2-7. Calculate Silhouette Score;
4-2-8. PCA for Visualization;
4-2-9. Dynamic consistently decides the optimal cluster number.

10. A machine learning system, characterized in that the system is constructed using the method described in claim 1.
